(19) United States
(12) Patent Application Publication
(10) Pub. No.: US 2008/0293083 A1
(43) Pub. Date: Nov. 27, 2008

(54) Method and Kit for Peptide Analysis

(75) Inventors: Bengt Bjellqvist, Uppsala (SE); David Fenyo, New York, NY (US); Jesper Hedberg, Uppsala (SE); Henrik Neu, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES AB, UPPSALA (SE)

(21) Appl. No.: 11/571,781

(22) PCT Filed: Jul. 5, 2005
(86) PCT No.: PCT/SE05/01113

§ 371 Date: Jan. 8, 2007

(30) Foreign Application Priority Data
Jul. 9, 2004 (SE) 0401854-5

(51) Int. Cl. C12Q 1/37; G01N 33/00; G06F 17/30

(52) U.S. Cl. 435/23; 436/86; 707/104.1; 707/E17.044

(57) ABSTRACT

The present invention relates to a method for peptide analysis, comprising the following steps: a) tagging N-terminals of peptides in sample(s) with mass tagging reagent(s) and mass balancing C-terminals of said peptides with mass balancing reagent(s), or vice versa; and b) mass spectrometry analysis of said peptides. The present invention also relates to a kit with global mass tagging reagents and mass balancing reagents for use in said method and a database with specific peptide information.

### FIELD OF THE INVENTION

The present invention is within the field of proteomics. More closely, the invention relates to a method for peptide analysis by global mass tagging of peptides. Preferably, the global mass tagging is used in combination with high resolution peptide separation for use in differential display.

### BACKGROUND OF THE INVENTION

The peptide based techniques presently used for differential analysis in proteomic studies normally contain the following steps: mass tagging, followed by digestion, ion exchange and/or some type of complexity reduction like ICAT (Isotope Coded Affinity Tags) disclosed in WO 00/11208 or COFRADIC (Combined Fractional Diagonal Chromatographic) method disclosed in WO 02/07716 combined with reversed phase chromatography (RPC) and finally identification and relative quantification with mass spectrometry (MS).

Global tagging aimed at relative quantification of peptides requires a technique independent of amino acid composition or posttranslational modifications, and is normally done after tryptic digestion of the proteins at the N- or C-terminal of the peptides [1,2,3]. Most commonly used is presumably acylation at the N-terminal at neutral pH with N-hydroxysuccinimide (NHS) ester, but at these conditions not only the N-terminal but also the 8-aminogroup of the lysines will react. To avoid the latter reaction, guanidation of the lysine group with O-methylisourea can be done prior to acylation [4]. An advantage of this approach is that this modification result in an increased ionisation efficiency for lysine containing peptides. Besides NHS-esters there exist a large number of other reagents possible to use with primary amino acids for example 2, 4 dinitrofluorobenzene [5], phenylisothiocyanate [6] or reaction with aldehyde followed by reduction [7]. Specific global tagging at the C-terminal has so far been done with ¹⁸O. Trypsin, chymotrypsin, lys-C and glu-C introduces up to two ¹⁸O atoms when the proteolysis is done in H₂¹⁸O. This enzymes will also catalyse the inclusion of two ¹⁸O atoms pro peptide in already digested peptide mixture at the positions corresponding to the cleavage sites of the enzyme used [8,9].

The use of internally balanced mass tags in global mass tagging has been described by Xzillion [10] and Applied Biosystems Institute [11]. In their approaches, the N-terminal participates in a reaction transferring a group containing a mass tagged low molecular weight reporter group as well as a group contributing to mass balance, where the reporter and mass balance are split apart in the fragmentation step.

A limitation with the above approach is that peptides with the same retention times and masses are contributing to the background signal, e.g., a mass peak originating from the tail of the isotope distribution of a peak with lower molecular weight or any peptide/protein present in low concentrations, will release the same low molecular reporter molecule. Therefore, the MS/MS signal from the peptide of interest will be indistinguishable from the MS/MS signal contributed from background noise. This will limit the possibility to make relative concentration determinations for low abundant peptides/proteins.

Isoelectric focusing (IEF) with immoblised pH gradients (IPG:s) is a method described within prior art. The technique used by Stephenson [12] results for the pH range 3.5-4.5 in 49 fractions with a width of 0.02 pH-units. This technique is dependent on the possibility to cut a IPG-strip in precise pieces within in a limited time frame to avoid diffusion. This handling is complicated and time consuming.

Thus, there is a need of an improved IEF technique and especially a separation technique that may be combined with global mass tagging of peptides for mass spectrometry (MS) analysis.

One further limitation in proteomics studies of today is that large parts of generated data in a data set are often redundant. This means that every time a proteomics study is performed the same proteins are quantified and identified several times regardless of their potential importance. For instance, in a classical 2D electrophoresis experiment the complete spot map is scanned and quantified even if only a few spots are of actual interest for the investigator. This generation of “unnecessary data” is very time consuming and it generates large bulks of data from which it is very cumbersome to extract meaningful information. Thus, in the current approaches the entire sample must be analysed. To avoid this, the existing workflow for proteomic studies must be improved to give the possibility to pre-select and study only the proteins/peptides of interest. To achieve this, information of the anticipated protein/peptide pattern from a particular sample must be known in advance, preferentially stored in a data base, and with this information a more intelligent experimental design can be developed. At the present, such an off-line pre-selection of proteins/peptides is not available.

### SUMMARY OF THE INVENTION

One object of the present invention was to enable relative concentration determinations of low abundant peptides/proteins in sample(s). The present invention enables this by providing a global mass tagging strategy, i.e. on that starts with digestion followed by tagging of N and/or C-terminal, and use of mass balancing groups to allow relative concentration to be determined in the MS/MS mode.

Another object of the invention was to provide a novel pre-MS separation technique with high resolution and reproducibility. According to the invention this is enabled by using isoelectric focusing in immobilised pH gradients as a step preceding RPC in the separation prior to MS.

A further object was to provide a novel way to select target protein sub-sets for proteome analysis by MS. For example, protein sub-sets constituting signalling pathways. This object is achieved by the invention by establishment of tryptic databases. The databases correspond to the characterised peptides originating from proteins present in complex samples like human sera, liver or brain. The database information should contain peptide composition including PTMs, identity of the corresponding gene and gene ontology assignments, but also an address to the peptide in a four dimensional analytical space given by the isoelectric point, the retention time in RPC, the peptide mass and the masses of fragment ions in the MS/MS spectrum.

Thus in a first aspect, the invention relates to a method for peptide analysis, comprising the following steps:
a) tagging N-terminals of peptides in sample(s) with tagging reagent(s) and mass balancing C-terminals of said peptides with mass balancing reagent(s), or vice versa; and
b) mass spectrometry analysis of said peptides.

As an alternative to N-terminal tagging, N-terminal arginines may be tagged.

The method of the invention is especially suitable for analysis of two samples which are differentially tagged. The first sample is provided with the light form of the reagent, i.e. with normal isotopes, and the second sample is provided with the heavy form of the reagent, i.e. with stable isotopes, for example deuterium and ¹³C. In this way, the samples may be compared or related to each other in a simultaneous analysis.

The samples may be complex samples which are enzymatically or chemically digested to generate peptides from proteins. Any endoprotease can be used for this purpose, such as LysC, ArgC, AspN, but preferably trypsin is used. For chemical digestion, for example cyanogens bromide may be used.

In the method according to the invention, the global tagging may be achieved in that the N-terminals are tagged with a low molecular weight mass tag reagent and the C-terminals are mass balanced with a mass balance reagent.

The present invention is especially useful for differential display of peptides in two different samples.

Preferably, the N-terminals of the peptides in one sample are tagged with heavy forms (such as D or ¹³C forms of) a reagent comprising N-acetoxysuccinimide, N-propoxysuccinimide, acetic anhydride, propionic anhydride, dinitrofluorobenzene, phenylisothiocyanate; or aldehyde for generation of alkyl or dialkyl derivative; and the C-terminal is enzymatically mass balanced with a reagent comprising ¹⁸O. The N- and C-terminals of peptides in the other sample are tagged and mass balanced with the light forms of the above reagents.

Alternatively, the method according to the invention achieves global tagging by tagging the C-terminals of the peptides in one sample with a low molecular weight mass tag reagent and mass balancing the N-terminals with a mass balance reagent. In this case, the C-terminal is preferably tagged with a reagent comprising ¹⁸O and the N-terminal is preferably mass balanced with heavy forms of (such as D or ¹³C forms of) a reagent comprising N-acetoxysuccinimide, N-propoxysuccinimide, acetic anhydride, propionic anhydride, dinitrofluorobenzene, phenylisothiocyanate; or aldehyde for generation of alkyl or dialkyl derivative. The C- and N-terminals of peptides in the other sample are tagged and mass balanced with the light forms of the above reagents.

The mass balancing of the C-terminals or N-terminals of the peptides is done either at the digestion or before mass spectrometry.

Preferably, step b) is preceded by a separation step and more preferably by a reverse phase chromatography, RPC, step. The RPC step may itself be preceded by a separation step. This could be one or more steps of chromatography, such as MDLC (multidimensional chromatography). Alternatively this could be isoelectric focusing, IEF, or a combination of chromatography and IEF.

Preferably, RPC is preceded by a IEF procedure. If a IEF procedure is used, it may be a one step or two step IEF procedure.

Preferably a two step procedure, wherein the first step is a liquid phase IEF and the second step is solid phase IEF with immobilised pH-gradients. The liquid phase IEF may be free flow electrophoresis, membrane separation (such as mini-IsoPrime), chromatofocussing or Sephadex IEF (13).

The second step may be repeated in a more narrow pH-range than used for the second step IEF. For easier handling, coloured pI markers may be included in the second step and any repetitions thereof.

The method may comprise an additional step c) collecting information about pI, retention time in RPC, peptide mass in MS and fragment ion mass in MS/MS for each peptide or sub-sets of peptides within a database.

If a database already is established, the method may comprise an additional step c) comparing pI, retention time in RPC, peptide mass and fragment ion mass in MS for each peptide or sub-sets of peptides with information in pre-established databases containing information about pI, retention time in RPC, peptide mass in MS and fragment ion mass in MS/MS for peptides of a proteome, or sub-set thereof.

In preferred embodiments, the invention relates to a method for peptide analysis, comprising the following steps:
a) sample preparation,
b) digestion of proteins to generate peptides,
c) tagging of N-terminals of peptides and mass balancing of C-terminals thereof, or vice versa;
d) high resolution separation of peptides of interest; and
e) subjecting said peptides to MS/MS.

For differential analysis, a preferred method of the invention comprises the following steps:
a) preparation of sample 1 and sample 2,
b) digestion of proteins to generate peptides,
c) tagging of N-terminals of peptides by reacting the N-terminals of peptides in sample 1 with a tag with the mass MN and the peptides in sample 2 with a tag with the mass (M_{N}+M_{add}) and mass balancing at the C-terminals thereof by reacting the C-terminals of the peptides of sample 1 with a reactant increasing the mass with (M_{C}+M_{add}) and the peptides of sample 2 with a tag increasing the mass with M_{C} or vice versa;
d) mixing sample 1 and sample 2,
e) performing a high resolution separation of the peptides in the resulting mixture, and
f) subjecting either all fractions or selected fractions resulting from said separation to a chromatographic (preferably reversed phase) separation followed by mass spectrometry where a relative quantification is done in the MS/MS spectra, relating the concentrations of a certain peptide in sample 1 to the concentration of the corresponding peptide in sample 2.

In contrast to prior art, global mass tagging according to the present invention is done through a reaction at the N-terminal and mass balance is created through a reaction at the C-terminal, or vice versa. Relative concentrations will be determined from the fragment ions in the MS/MS spectrum. However, these fragment ions will differ from the fragment ions generated from other peptides appearing at the same mass in the primary MS. Provided that the mass of a peptide of interest is known, as well as the masses of the fragment ions resulting from this peptide, it will be possible to collect ions with the mass (M_{peptide}+M_{N}+M_{C}+M_{add}) also in the cases when no mass peak is detectable in the primary spectrum. In the resulting MS/MS spectrum the relative concentrations of the peptide in sample 1 and 2, respectively, can be determined from the relative intensities of the peaks appearing as doublets differing in mass with M_{add} mass-units at positions known to correspond to the masses of the fragment ions generated from the peptide of interest.

To maximize the dynamic range possible to cover, the present invention also relates to a peptide database for the sample type used. Besides the origin and composition of the peptides this data base should give addresses to the peptides in a four dimensional space given by the isoelectric point, the retention time in RPC, the peptide mass and the masses of the fragments ions appearing in the MS/MS spectrum. The data in the database should be generated with peptides tagged at both the N and C-terminal with the reagents transferring the masses MN and M_{C} to the terminals. In the generation of the data base it is clearly advantageous to use the highest resolution feasible in the steps preceding MS.

For differential analysis, another preferred method of the invention comprises the following steps:
a) preparation of sample 1 and 2,
b) digestion of proteins to generate peptides,
c) tagging of N-terminals of peptides by reacting the N-terminals of peptides in sample 1 with a tag with the mass MN and the peptides in sample 2 with a tagg with the mass (M_{N}+M_{add}),
d) mixing of sample 1 and 2,
e) performing a high resolution separation of the peptides in the resulting mixture,
f) subjecting, either all fractions, or selected fractions resulting in said separation to a chromatographic (preferably reversed phase) separation,
g) after the mixing in step d) but prior to the relative quantification, the C-terminals of the peptides in the mixed sample are reacted with a mixture containing two isotopic variants of the reactant transferring the mass M_{C} or the mass (M_{C}+M_{add}) to one or two reactive positions present at the C-terminal, and
h) quantification is done in the MS/MS spectra, relating the concentrations of a certain peptide in sample 1 to the concentration of the corresponding peptide in sample 2 by selecting in the primary spectra a mass-peak containing peptides, which in the case of one reactive group at the C-terminal has been reacted to get a mass increase of (M_{N}+M_{C}+M_{add}) or in the case of two reactive groups at the C-terminal has been reacted to get a mass increase of (M_{N}+2*M_{C}+M_{add}) and/or (M_{N}+2*M_{C}+2*M_{add}).

If the peptide to be analysed initially has a mass equal to M_{peptide} and if, in order to simplify the situation it is assumed that this peptide is present in equal amounts in sample 1 and sample 2, the result after mixing of samples will be a mixture containing equal amounts of the peptide with the masses (M_{peptide}+M_{N}) and (M_{peptide}+M_{N}+M_{add}) respectively. This mixture is than reacted with a reactant mixture containing two isotopic variants of a reactant transferring to the C-terminal of the peptides the masses M_{C} and (M_{C}+M_{add}), respectively. If, again of simplicity reasons, it is assumed that the reactant mixture contain equal amounts of the isotopic variants of the reactant, the peptide of interest will, in the finally resulting mixture be present with three different masses: (M_{N}+M_{C}) representing 25% of the peptide, (M_{N}+M_{C}+M_{add}) representing 50% of the peptide and (M_{N}+M_{C}+2 M_{add}) representing 25% of the peptide. The peak with the mass (M_{peptide}+M_{N}+M_{add}) selected for generation of a MS/MS spectrum will with the assumptions made contain equal amounts of peptide with the additional mass bound to the N-terminal and C-terminal group, respectively. In the resulting MS/MS spectra the mass peaks relating to the generated fragments will appear as doublets differing in mass with M_{add} mass units. When the value M_{add} is small (1-5 mass units) the peak selected in the primary spectrum will not only contain peptides with the mass generated by adding the mass M_{add} in the reaction with either the N- or C-terminal, but also peptides with only the masses M_{N} and M_{C} added in the reaction and the mass M_{add} contributed by heavy isotopes (mainly ¹³C and ³⁴S) originally present in the peptide. This will cause the peak ratio in the doublets in the MS/MS spectrum to slightly deviate from a 1:1 ratio in the case, when the two samples contain identical amounts of the peptide of interest and the reactant mixture contain equal amounts of the isotopic variants of the reactant used for mass balancing. With the identity of the peptide known and the composition of the fragment corresponding to a peak doublet known, it is from a determination of the ratio between the two peaks in the doublet easy and straight forward to relate the concentration of the peptide in sample 1 to the peptide concentration in sample 2 provided that the ratio of the isotopic variants used in the mass balancing step is known.

This alternative approach contains some obvious complications in relation to the first design described for differential analysis. Firstly, the primary mass spectra as well as the secondary MS/MS spectra become more complicated and secondly only 50% of the peptides originally present are used in the quantification. However, the alternative approach also offers a number of important advantages:

1/As a result of isotopic effects, especially when hydrogen to deuterium is used for generation of mass differences, differently tagged peptides can fail to co-elute from an RPC column [12]. Similarily the behaviour of the isoforms might differ in separation techniques preceding the RPC. Mixing of the samples immediately after the mass tagging and performing the mass balancing step after the separation causing problems, will allow the use of cheaper mass balancing reactants based on hydrogen to deuterium exchange.
2/Products resulting from the mass balancing step could be unstable and fall apart in reactions in the separation steps preceding the MS. Example are the type of non-covalent complexes possible to generate between organic sulphonic acids and arginine/homoarginine for example the complex generated between naphthalene-disulfonic acid and arginine which survives in MS, but which can not be expected to survive in a possible preceding isoelectric focusing step.
3/The change of conditions between the tagging step and the mass balancing step could introduce a risk for peptide losses. When enzymatic catalysis is to be used for the introduction of ¹⁸O at the C-terminal of tryptic peptides, there is, with the technique initially described, a need to evaporate the sample to dryness prior to the addition of H₂ ¹⁸O. Re-dissolution of peptides depend on sequence and give a very pronounced risk for peptide losses. This alternative approach does not require re-dissolution.
4/One of the isotopic variants used for mass balancing will in many cases be expensive as for example H₂ ¹⁸O. The sample will be split in many fractions prior to RBC and MS/MS. In most cases only a limited number of these fractions will be used for quantification with MS/MS. Consumption of expensive reagents can be minimized by mass balancing only the fractions to be used in MS/MS.

For differential analysis, a further preferred method of the invention comprises the following steps:
a) preparation of sample 1 and 2,
b) digestion of proteins to generate peptides,
c) tagging of N-terminals of peptides by reacting the N-terminals of peptides in sample 1 with a tag with the mass MN and the peptides in sample 2 with a tag with the mass (M_{N}+2),
d) mixing of sample 1 and 2,
e) performing a high resolution separation of the peptides in the resulting mixture,
f) subjecting, either all fractions, or selected fractions resulting in said separation to a chromatographic (preferably reversed phase) separation, and
g) after the mixing in step d) but prior to the relative quantification, addition of H₂ ¹⁸O together with an enzyme catalysing oxygen exchange between water and the C-terminal carboxyl oxygens. (As the peptides after the H₂ ¹⁸O addition are solubilised in a H₂ ¹⁸O/H₂ ¹⁶O mixture the result is that 0, 1 or 2 are transferred to the C-terminal of the peptides in the mixture.) and
h) quantification is done in the MS/MS spectra, relating the concentrations of a certain peptide in sample 1 to the concentration of the corresponding peptide in sample 2 by selecting in the primary spectra a mass-peak containing peptides reacted to give a mass increase of (M_{N}+2) and/or (M_{N}+4).

In a second aspect, the invention relates to a kit with tags for differential display, comprising: mass tags and mass tag balancing groups.

In a preferred embodiment, the kit comprises N-acetoxysuccinimide+(¹³Cₙ and/or Dₙ) N-acetoxysuccinimide+H₂¹⁸O, wherein n=2 or 4.

The kit may also comprise N-propoxysuccinimide+(¹³Cₙ and/or Dₙ) N-propoxysuccinimide+H₂¹⁸O, wherein n=2 or 4.

In another preferred embodiment, the kit comprises acetic anhydride+(¹³Cₙ and/or Dₙ) acetic anhydride+H₂¹⁸O, wherein n=2 or 4.

The kit may also comprise propionic anhydride+(¹³Cₙ and/or Dₙ) propionic anhydride+H₂¹⁸O, wherein n=2 or 4.

In a further preferred embodiment, the kit comprises formaldehyde+¹³C and/or D formaldehyde+¹⁸O, wherein n=2 or 4.

Any other aldehyde may also be used.

Furthermore, light and heavy forms of dinitrofluorobenzene and phenylisothiocyanate may also be used [5][6].

Optionally, the kit also comprises trypsin.

In a third aspect, the invention relates to a database comprising information about the origin and composition of the peptides as well as isoelectric point, retention time in RPC, peptide mass and the masses of the fragments ions appearing in the MS/MS spectrum. The database is preferably arranged in accordance with the method of collecting information about pI, retention time and MS data as described above.

An advantage of global mass tagging, compared to more selective tagging, is that differential display no longer is limited to a few peptides per protein. When compared chemistries tagging at only selected residues, for example at methionine residues, the global approach will, for peptides of adequate size, give an increase of the number of tagged peptides with a factor 5. Tagging of cysteinyl residues instead of methionyl residues gives an even smaller number of tagged peptides per protein. Thus, use of balanced mass tags according to the invention will increase the dynamic range within which differential display successfully can be used. Another advantage is that global tagging according to the invention increases the chance to make measurements on peptides close to N- and C-terminal to control if an observed concentration difference relates to the full-length protein. Similarly there will be increased possibilities to check the importance of posttranslational modifications (PTMs) or alternative splicing at the site of interest.

A further advantage of global mass tagging according to the invention is that it can accept some incomplete digestion as well as some peptides resulting from chymotryptic activity.

### BRIEF DESCRIPTION OF THE DRAWING

FIG. 1 shows a schematic overview of an exemplifying way to perform the method of the invention.

### DETAILED DESCRIPTION OF THE INVENTION

In the method of the invention several different reagents may be used for mass tagging at the N-terminal. Examples of useful mass tagging reagents are: N-acetoxysuccinimide, N-propoxysuccinimide, propionic anhydride, formaldehyde, or other aldehydes, for generation of dimethyl derivative by reductive amination. For differential tagging the light reagents contain the normal isotopes and the heavy reagents are substituted with deuterium (Dₙ) or are alternatively substituted with ¹³Cₙ, wherein n is a number from 1-4 depending on the chosen reagent.

One way to balance the N-terminal mass caused by the mass tag is to use trypsin to include ¹⁶O and ¹⁸O at the C-terminal either in connection with the tryptic digestion [8] or at a later stage where then trypsin is included together with mass tagged sample peptides in a 1/1 mixture of H₂¹⁶O/H₂¹⁸O to catalyse the ¹⁶O/¹⁸O exchange.

Other ways of N-terminal tagging are reactions at N-terminal lysines or conversion of lysine to homoarginine followed by the use of reactants with specificity for arginine/homoarginine. This is useful when trypsin is used for digestion of the proteins.

As mentioned above, the mass tagging may also be at the C-terminal in which case the mass balancing is at the N-terminal.

To fully utilise global mass tagging, complex samples will require a separation method with very high resolving power. According to the present invention, use is preferably made of isoelectric focusing with immobilised pH-gradients (IPG:s). Preferably, a two-step procedure with IEF in liquid phase and then IEF with IPG is used in a narrower pH range than in the first step. Besides the high resolving power this technique gives a number of other advantages of which the predictability and the high reproducibility is of special importance in the present context. The predictability is of great value as it limits the efforts required for the localisation of the IPG fraction within which a peptide of interest has focused.

The first focusing step can be run in a number of different ways, as examples either in a polyacrylamide gel strip containing a wide range IPG pH 3-10, or with conventional preparative isoelectric focusing carrier ampholyte in combination with either Sephadex [13] or in solution in a chamber apparatus of the type described of Zuo and Speicher [14]. Any of these approaches will allow a prefractionation in 5 to 20 narrow pH range peptide fractions. In a second step these fraction can be separated either in narrow range IPG-strips as described by Stephenson or alternatively in the chamber equipment described by Zuo and Speicher, but then equipped with IPG membranes within the pH range of the peptide fraction to be separated, and with neighbouring membranes differing in pH with only 0.01-0.02 pH-units.

Databases of tryptic peptides comprising information about the behaviour of the peptides in IPG-focusing, RPC and tandem MS will be useful for several applications. Peptide databases with peptide identities and positions in a four dimensional space given by pI, retention time, peptide mass and fragment mass in the MS/MS spectrum will allow standardised methods to be used, not only for concentration determinations, but also for localisation of alternative splicing sites or PTM:s related to disease. In a longer perspective this type of peptide database containing all the information required for the characterisation and concentration determination of the peptides can be seen as a first step towards analytical methods useful in personalised medicine.

The combined use of global mass tagging according to the invention and IEF connected to peptide databases is expected to reach a much larger and more diversified use than traditional tagging, 2-D electrophoresis and/or MDLC followed by MS.

### Experimental Part

The invention will now be described in association with some non-limiting examples.

Beneath mass tagging is described at the N-terminal preferably with the aid of NHS ester, transferring a N-terminal mass tagging reagent, see above, containing either none (for light reagent) or two deuterium atoms (for heavy reagent). Balancing the mass of the tagged and untagged peptides is not done in connection with the digestion, but catalytically prior to RPC and MS with the aid of trypsin in water containing ¹⁶O/¹⁸O in the ratio 1/1. For a peptide present in equal amounts in sample and reference a 1:3:3:1 intensity distribution will result for the peaks with the masses M, M+2, M+4 and M+6, respectively.

### 1. Generation of Peptide Data

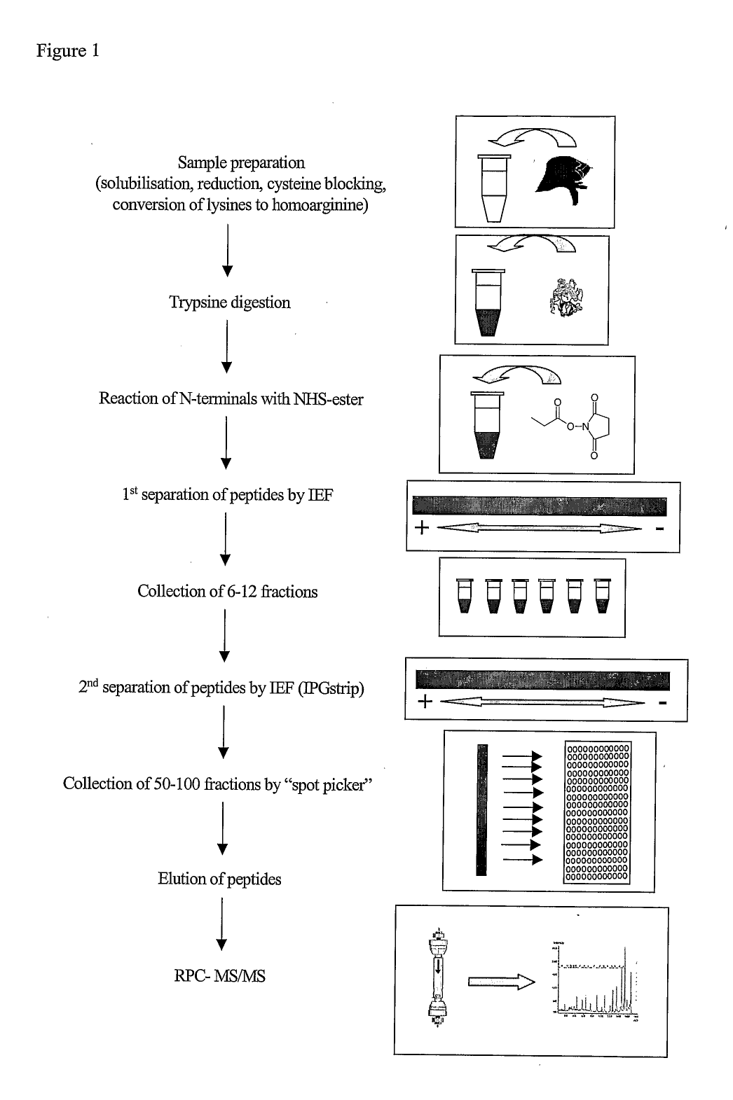

If a relevant database is not already available, the first step will be to generate the data required for the peptide database covering the samples to be used in later differential display experiments. For these experiments a reference sample is used which might be a mixture of samples covering different condition of biological relevance. The following experimental steps are involved, see FIG. 1:
1. Sample preparation (solubilisation, denaturation, reduction, protection of cysteinyl residues with DeStreak™ or alkylating agent. Conversion of lysines to homoarginine [8]).
2. Trypsin digestion.
3. Reaction with the type of NHS-ester later to be used for differential display.
4. Separation of the peptides in complex samples in a two step IEF procedure with IPG-focusing in the second step into the number of fractions (100, 300, 600 or 1200) judged to be needed based on the sample complexity. For a less complex sample, a one-sep procedure may be sufficient.
5. Identification of peptides in the different IEF-fractions with RPC followed by MS/MS.
6. Compile the accumulated information relating to the peptides including pI, retention time, peptide mass and masses of fragment ions in the MS/MS spectra in a database.

### Peptide Database Covering a Human Proteome

The human genome corresponds to 30-40.000 expressed genes. Tryptic digestion of the products resulting from one gene is expected to give on the average 40 peptides in a M_{w} range suitable for MS detection (mean Mw of a protein of 50 kDa gives 25-30 peptides, alternative splicing and PTM:s adding additionally 10-15 peptides, or totally the genome corresponds to 1.2-1.6 million peptides). In a complex tissue sample it is conceivable that as much as 75% of these genes are expressed.

To generate a database, the prerequisite is that these peptides can be separated in fractions suitably sized for use in MS preceded by RPC and that only a limited number of the peptides are present in more than one of the resulting fractions. The present invention fulfils this prerequisite.

The peptides should be characterised by the identity of the corresponding gene, their composition including PTM:s, the gene ontology assignments (GO) valid for the corresponding gene products and their position as mass tagged peptides in a four dimensional analytical space given by their pI-values, retention times, peptide masses and the fragment masses in the MS/MS spectra. For information on gene ontology assignments see [http://www.ebi.ac.uk/GOA/HUMAN_release.html].

### 2. Differential Display

For differential display experiments, samples and reference are in the three first steps treated in parallel but separately. The following experimental steps are involved, see FIG. 1:
1. Solubilisation, denaturation and reduction of samples. Protection of cystenyl residues with DeStreak™ or alkylating agent. Convert lysine to homoarginine.
2. Trypsin digestion of samples as well as reference.
3. Reaction of samples with NHS-ester containing no D (light reagent), reaction of reference with NHS-ester containing two D (heavy reagent) atoms transferred to the peptide in the reaction.
4. Mixing of samples with reference. Separation in liquid phase IEF to split the peptides in the sample-reference mixtures in 6 to 12 fractions. Selection of pH interval for initial use. Remaining fractions frozen and stored for possible future use.
5. Peptides in selected pH-intervals re-focused in narrow range IPG strip. Gel on focused IPG strips divided (by a spot picker or other cutting equipment) in 50-100 parts and transferred to micro-titre plate. Elution of peptides from gel pieces.
6. Selection of samples for initial use with RPC and MS. Catalytic inclusion of ¹⁶O/¹⁸O in the ratio 1/1 into selected samples. Generations of list of peptides to be compared in differential display. Programming of mass spectrometer instrument with retention times and masses for the peptides to be compared. Peak corresponding to the mass M+2 and/or M+4 to be selected for generation of MS/MS spectra.
7. Evaluation of first set of runs with differential display. Selection of new set of samples to run. Generation of new list of peptides and so on, until the desired information has been collected.

The tagging and mass balancing approach according to the invention is possible to realize with several well described specific reactions. It can be expected that the tagging will give high specificity and very low background noise. The other global mass tagging alternatives described by Xzillion [10] and more recently by ABI [11] are based on compounds, also intended for reaction at the N-terminal, but with the mass balance originally incorporated in the tag, detached in collisions prior to the generation of the MS/MS spectrum and used as reporter molecule in the resulting spectra. The most important advantage of the present invention is that it will allow the coverage of a much wider dynamic range. Another advantage is that the reagents used are cheaper and easier to synthesise.

### 3. Separation Technique Characterised of High Resolution and Reproducibility

To cover all tryptic peptides a pre-focusing step, followed by focusing in a number of narrow range gradients is required. A preferred liquid IEF pre-focusing step may be performed in a mini-IsoPrime with the focusing done in the liquid phase as this give an easy transfer to the next focusing step. For example, the technique can be used with a membrane unit for the first focusing step and with IPG strips for the second focusing step. The use of 5-6 narrow range IPG strips, with pH ranges suitably adjusted to the application should allow samples to be split in 250-300 well resolved fractions.

While focusing with IPG:s will give the resolution required, this is by itself not enough for the suggested application. There is also a need to reproducibly collect comparable fractions with a width of 0.02, 0.01 or 0.005 pH-units. According to the present invention, the cutting of the IPG strip, alternatively the collection of gel fractions from the strip, is preferably done in relation to the positions of focused bands generated with the aid of coloured pI markers. For example, the required type of pI markers can be produced through the reaction of CyDyes™ with cysteine containing peptides, where the peptides have been designed to have the pI value required. Equipment for automatic gel collection/strip cutting should be able to determine the midpoint of the focused band with a precision of approx. 0.1 mm, which should be adequate for generation of as well 5 as 2.5 mm wide fractions.

The high resolution achieved with isoelectric focusing combined with RCP, the fact that the masses to be used for generation of MS/MS spectra are known and that the measurements are made in the MS/MS spectra, should allow relative concentration determinations down to the low femtomole range. Combining this with the high peptide loads possible to use in IPG focusing (10-20 mg peptides) indicates that mass balanced labelling in combination with IPG focusing should give a dynamic range for differential display corresponding to 10⁶.

### REFERENCES

1. Hsu J-L, Huang S—Y, Chow N—H and Chen S-H, Stable-isotope Dimethyl labeling for quantitative Proteomics Anal. Chem. 2002, 75, 6843-6852
2. Liu P and Regnier F E, An Isotope coded strategy for Proteomics Involving Both Amine and Carboxyl Group labelling. J. Proteome Res. 2002, 1, 443-450
3. Chakraborty A and Regnier F E, Global internal standard technology for comparative proteomics J Chromatogr A 2002, 949, 173-184
4. Chen, X. H.; Chen, Y. H.; Anderson, V. E., Protein cross-links: universal isolation and characterization by isotopic derivatization and electrospray ionization mass spectrometry, Anal. Biochem. 1999, 273, 192-203.
5. Zhang, X.; Jin, Q. K.; Carr, S. A. Annan, R. S., N-terminal peptide labeling strategy for incorporation of isotopic tags: a method for the determination of site-specific absolute phosphorylation stoichiometry, Rapid. Commun. Mass Spectrom. 2002, 16, 2325-2332.
6. Mason, D. E.; Liebler, D.C., Quantitative analysis of modified proteins by LC-MS/MS of peptides labeled with phenyl isocyanate, J. Proteome. Res. 2003, 2, 265-272.
7. Zhang, H.; Bart, B. M.; Eng, J.; Aebersold, R. In Mass Spectrometry and Allied Topics, 2003.
8. Schnolzer M, Jedrzejewski P and Lehmann W D,
   Protease-catalyzed incorporation of 18O into peptide fragments and its application for protein sequencing by electrospray and matrix-assisted laser desorption/ionization mass spectrometry. Electrophoresis. 1996 May; 17(5):945-53
9. Yao, X.; Afonso, C.; Fenselau, C. J., Dissection of proteolytic 18O labeling: endoprotease-catalyzed ¹⁶O-to-¹⁸O exchange of truncated peptide substrates, Proteome Res. 2003, 2, 147-152.
10. Thompson A, Schafer J, Kuhn K, Kienle S, Schwarz J, Schmidt G, Neumann T and Hamon C Tandem mass tags: a novel quantification strategy for comparative analysis of complex protein mixtures by MS/MS. Anal Chem. 2003 Apr. 15; 75(8): 1895-904.
11. www.appliedbiosystems.com/itraq
12. Cargile B J, Bundy L J, Freeman T W and Stephensson J L
   Gel based isoelectric focusing of peptides and the utility of isoelectric point in protein identification. J Proteome Res. 2004 January-February; 3(1): 112-9.
13 Gorg A, Boguth G, Kopf A, Reil G, Parlar H, Weiss W. Related Articles, Links Sample prefractionation with Sephadex isoelectric focusing prior to narrow pH range two-dimensional gels. Proteomics. 2002 December; 2(12):1652-7
14 Zuo X, Speicher D W. Related Articles, Links
   Microscale solution isoelectrofocusing: a sample prefractionation method for comprehensive proteome analysis. Methods Mol Biol. 2004; 244:361-75

1: A method for peptide analysis, comprising the following steps:
a) tagging N-terminals of peptides in sample(s) with tagging reagent(s) and mass balancing C-terminals of said peptides with mass balancing reagents(s), or vice versa; and
b) mass spectrometry analysis of said peptides.

2: The method of claim 1, wherein there are two samples which are differentially tagged.

3: The method of claim 1, wherein the sample(s) are complex sample(s) which are enzymatically or chemically digested to generate peptides from proteins.

4: The method of claim 3, wherein the digestion is with trypsin.

5: The method of claim 1, wherein the N-terminals are tagged with a low molecular weight mass tag reagent and the C-terminals are mass balanced with a mass balance reagent.

6: The method of claim 2, wherein the N-terminals of peptides in one sample are tagged with heavy forms (D and/or ¹³C forms of) a reagent comprising N-acetoxysuccinimide, N-propoxysuccinimide, acetic anhydride, propionic anhydride, 2,4 dinitrofluorobenzene phenylisothiocyanate; or aldehyde for generation of alkyl or dialkyl derivative; and the C-terminals are enzymatically mass balanced with a reagent comprising ¹⁸O, and wherein the N- and C-terminals of peptides in the other sample are tagged and mass balanced with the light forms of the above reagents.

7: The method of claim 1, wherein the C-terminals are tagged with a low molecular weight mass tag reagent and the N-terminals are mass balanced with a mass balance reagent.

8: The method of claim 2, wherein the C-terminals of peptides in one sample are tagged with a reagent comprising ¹⁸O and the N-terminals are mass balanced with heavy forms (D and/or ¹³C forms of) a reagent comprising N-acetoxysuccinimide, N-propoxysuccinimide, acetic anhydride, propionic anhydride, 2,4 dinitrofluorobenzene, phenylisothiocyanate; or aldehyde for generation of alkyl or dialkyl derivative, wherein the C- and N-terminals of peptides in the other sample are tagged and mass balanced with the light forms of the above reagents.

9: The method of claim 3, wherein the C-terminals or N-terminals of the peptides are mass balanced either at the digestion or before mass spectrometry.

10: The method of claim 1, wherein step b) is preceded by a separation step.

11: The method of claim 1, wherein step b) is preceded by a reverse phase chromatography, RPC, step.

12: The method of claim 11, wherein the RPC step is preceded by a separation step.

13: The method of claim 12, wherein the separation is by one or more steps of chromatography.

14: The method of claim 12, wherein the separation is by isoelectric focusing, IEF.

15: The method of claim 14, wherein the IEF is a two step IEF procedure.

16: The method of claim 15, wherein the first step is a liquid phase IEF and the second step is solid phase IEF with immobilised pH-gradients.

17: The method of claim 15, wherein the second step is repeated in a more narrow pH-range than used for the second step IEF.

18: The method of claim 15, wherein coloured pI markers are included in the second step and any repetitions thereof.

19: The method of claim 1, comprising a further step c) collecting information about pI, retention time in RPC, peptide mass in MS and fragment ion mass in MS/MS for each peptide or sub-sets of peptides within a database.

20: The method of claim 1, comprising a further step c) comparing pI, retention time in RPC, peptide mass in MS and fragment ion mass in MS/MS for each peptide or sub-sets of peptides with information in pre-established databases comprising information about pI, retention time in RPC, peptide mass in MS and fragment ion mass in MS/MS for peptides of a proteome, or sub-set thereof.

21: A kit with tags for differential display, comprising:
mass tags reagents and mass tag balancing reagents.

22: The kit of claim 21, comprising N-acetoxysuccinimide+(¹³Cₙ and/or Dₙ) N-acetoxysuccinimide+H₂¹⁸O, wherein n=2 or 4.

23: The kit of claim 21, comprising acetic anhydride+(¹³Cₙ and/or Dₙ) acetic anhydride+H₂¹⁸O, wherein n=2 or 4.

24: The kit of claim 21, comprising formaldehyde+¹³C and/or D formaldehyde+¹⁸O, wherein n=2 or 4.

25: The kit of claim 21, also comprising trypsin.

26: A database arranged in accordance with claim 19.
